(19) United States
(12) Patent Application Publication
(10) Pub. No.: US 2002/0099284 A1
(43) Pub. Date: Jul. 25, 2002

(54) Method and apparatus for characterizing a location at an examination subject

(76) Inventor: Klaus Herrmann, Nuernberg (DE)

Correspondence Address: SCHIFF HARDIN & WAITE, 6600 SEARS TOWER, 233 S WACKER DR, CHICAGO, IL, 60606-6473, US

(21) Appl. No.: 09/993,176

(22) Filed: Nov. 19, 2001

(30) Foreign Application Priority Data
Nov. 17, 2000 (DE) 10057027.5

(51) Int. Cl. A61B 5/05

(52) U.S. Cl. 600/407

(57) ABSTRACT

In a method and an apparatus which allow a location at an examination subject to be characterized, a volume dataset of a subject is generated, and an image is generated from the volume dataset, and a location in the image is marked to identify a location at the examination subject, represented in the image. Based on the mark that has been set in the image, a location characterizing unit adjusted relative to the subject so as to characterize a location at the subject which substantially corresponds to the marked location in the image.

### BACKGROUND OF THE INVENTION

1. Field of the Invention

The present invention is directed to a method and to an apparatus for the characterization of a location at an examination subject.

2. Description of the Prior Art

The employment of, for example, nails, screws and Kirschner wires for fixing a broken bone is standard in surgery (see, for example, “Pschyrembel Klinisches Wörterbuch”, Berlin, N.Y., de Gruyter, 1990, 256^{th} Edition, page 375). An attending physician can, for example, plan the positioning of the nail, the screw or the Kirschner wire at the broken bone on the basis of X-ray images of the broken bone and can characterize the entry point and angle of the nail, the screw or the Kirschner wire at the patient. The physician, however, has not guidance other than the X-ray images.

German OS 198 07 884 also discloses a method and an apparatus wherein intra-operatively acquired exposures of an examination subject are compared to preoperatively and/or intra-operatively produced exposures in order to determine and visualize the relative position of the examination subject relative to a surgical instrument.

### SUMMARY OF THE INVENTION

An object of the present invention is to provide a method and an apparatus such that pre-conditions that improve the quality of the characterization at the patient are created with it or with its assistance.

This object is inventively achieved in a method for characterizing a location at an examination subject having the following steps:

a) Generating a volume dataset of the examination subject;

b) Generating an image from the volume dataset;

c) Marking a location in the image; and

d) Based on the marking that has been set in the image, adjusting a unit for characterizing a location at the examination subject so that the location characterized at the examination subject that substantially corresponds to the location marked in the image.

A volume dataset thus can be generated of, for example, a broken bone of a patient. According to one embodiment of the invention, the volume dataset can be produced with an X-ray system. According to a version of the invention, further, the volume dataset is generated by a drive that encompasses an electric motor. The volume dataset also can be produced by automatic movement with a drive or drives. Subsequently, images of the broken bone are generated from the volume dataset, the positioning of the nail, the screw or the Kirschner wire for fixing the broken bone being planned therewith. For this purpose, a location that corresponds to the future position of the nail, screw or Kirschner wire at the broken bone is inventively marked in the image. Based on the location marked in the image, that location at the patient at which the nail, screw or Kirschner wire is to be introduced is subsequently characterized so that the nail, screw or Kirschner wire is arranged at the location at the broken bone that corresponds to the marked location in the image. The physician thus has an especially practical method for finding the exact entry point and/or the angle of the nail, of the screw or of the Kirschner wire at the patient, so that the quality of the treatment of the patient is enhanced.

According to one version of the invention, the volume dataset is generated by a computer that generates the image from the volume dataset.

In a further embodiment of the invention the image is a 2D image or a 3D image. Different images of the bone are thus available to the physician and the physician can select the images that seem most beneficial therefrom.

According to one version of the invention, the image can be displayed on a viewing device and marking can be set by a track ball, a joystick and/or a light pen. An embodiment of the invention is especially advantageous wherein the marking is set by a touch screen since the viewing device and the marking setting unit can thus be combined in one device and the number of devices is reduced. Moreover, a touch screen is especially simple to use and can be relatively easily sterilized, which is particularly important since the invention is also provided for medical purposes and, in particular, for use in an operating room.

In another version of the invention arrangement for characterizing a location at the examination subject is movable with a drive. The drive can be an electric motor. The characterization at the examination subject is especially exact when the arrangement for characterizing the location is automatically aligned at the examination subject with the drive.

In a further embodiment of the invention, the location at the examination subject is characterized by an optical sighting device that, according to a further version of the invention, characterizes the location at the examination subject with an optical beam. This is preferably a laser light sighting device since a laser beam emitted by the laser light sighting device is highly visible, and thus the physician can especially easily recognize the characterized entry point and/or angle at the patient.

The arrangement for generating a volume dataset can be a C-arm that, according to one embodiment, is movable relative to its angulation axis and/or orbital axis and, in particular, is adjustable with at least one drive, which preferably is an electric motor. For easy operation, the C-arm is automatically moved with the drive or drives.

In one embodiment of the invention the arrangement for characterizing a location at the examination subject is arranged at the C-arm, and the C-arm can move automatically such that the location at the patient is automatically characterized.

The above object is also achieved in an apparatus having an arrangement for generating a volume dataset, an arrangement for generating an image from the volume dataset, an arrangement for setting a marking in the image and an arrangement for characterizing a location at the examination subject that substantially corresponds to the location marked in the image. Inventively, the apparatus is designed such that a volume dataset of, for example, a broken bone is produced from which an image of the bone wherein a marking can be set can in turn be generated. The marking in the image corresponds to the future position of a nail, a screw or a Kirschner wire for fixing the broken bone. Moreover, the inventive apparatus is fashioned such that, based on the marking in the image, the arrangement for characterizing a location at the examination subject characterizes the entry point and/or the angle of the nail, screw or Kirschner wire at the patient.

### DESCRIPTION OF THE DRAWINGS

FIG. 1 is a side view of a C-arm X-ray apparatus operating according to the invention.

FIG. 2 shows a portion of a broken shin bone.

FIG. 3 shows an image of the broken shin bone.

### DESCRIPTION OF THE PREFERRED EMBODIMENTS

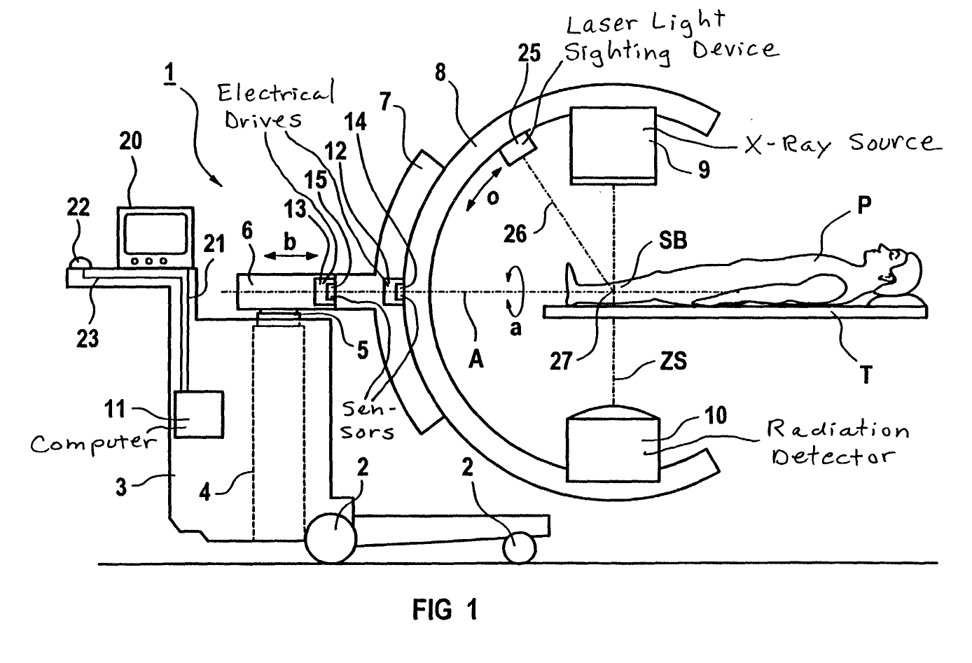

FIG. 1 schematically shows a side view of a C-arm X-ray apparatus 1. The C-arm X-ray apparatus 1 has an apparatus carriage 3 movable on wheels 2. The C-arm X-ray apparatus 1 has a lifting mechanism 4 with a column 5 schematically indicated in FIG. 1. A holder 6 is arranged at the column 5, a support 7 for a C-arm 8 being in turn arranged at the holder 6. The C-arm 8 carries an X-ray source 9 and an X-ray detector 10 that are arranged opposite one another at the C-arm 8 so that a central ray ZS of an X-ray beam emanating from the X-ray source 9 strikes the detector surface of the radiation detector 10 approximately centrally.

In a known way, the support 7 is seated at the holder 6 so as to be rotatable around a common axis A of the holder 6 and the support 7 (see double arrow ‘a’, angulation) and can be displaced in the direction of the axis A (see double arrow ‘b’). Along its circumference, the C-arm 8 is seated to be displaceable in the direction of the double arrow ‘o’ at the support 7 relative to the support 7 (orbital motion).

The C-arm 8, which is connected to the column 5 of the lifting mechanism 4 via the support 7 and the holder 6, is vertically adjustable relative to the device carriage 3.

A patient P schematically shown in FIG. 1 lies on a table T that is likewise only schematically indicated and is transparent for X-radiation, and is vertically adjustable with a lifting mechanism (not shown). As a result of the aforementioned adjustment possibilities of the C-arm X-ray apparatus 1 and the table T, the patient P can be radiologically examined a large variety of ways, whereby the X-rays with the central ray ZS emanating from the X-ray source 9 penetrating the patient P and strikes the radiation detector 10.

The C-arm X-ray apparatus 1 is particularly provided for producing a volume dataset of body parts of the patient P. In the exemplary embodiment, a computer 11 is arranged in the device carriage 3, the computer 11 being connected to the radiation detector 10 (in a way that is not shown) and reconstructing a volume dataset of the body part to be displayed in a known way from a series of 2d projections acquired with the X-ray source 9 and the radiation detector 10. These images are acquired with an adjustment of the C-arm around a body part of the patient P to be displayed in an image. The C-arm is adjusted by about 190° along its circumference in the direction of the double arrow ‘o’ relative to the support 7 or relative to the angulation axis A, with approximately 50 through 100 2D projections being acquired during the adjustment. In the present exemplary embodiment, the computer 11 controls the adjustment of the C-arm 8 with an electrical drive 12 arranged in the support 7 or with an electrical drive 13 arranged in the holder 6. The computer 11 is connected to the electrical drives 12 and 13 in a way that has not been shown.

In order to reconstruct the volume dataset from the series of 2D projections, respective distance sensors 14 and 15 are integrated in the electrical drives 12 and 13. The sensors 14 and 15 allocate a position of the C-arm 8 relative to the body part to be displayed for each of the 2D projections of the body parts to be registered. Finally ,projection geometries that are required for the reconstruction are determined from the positions.

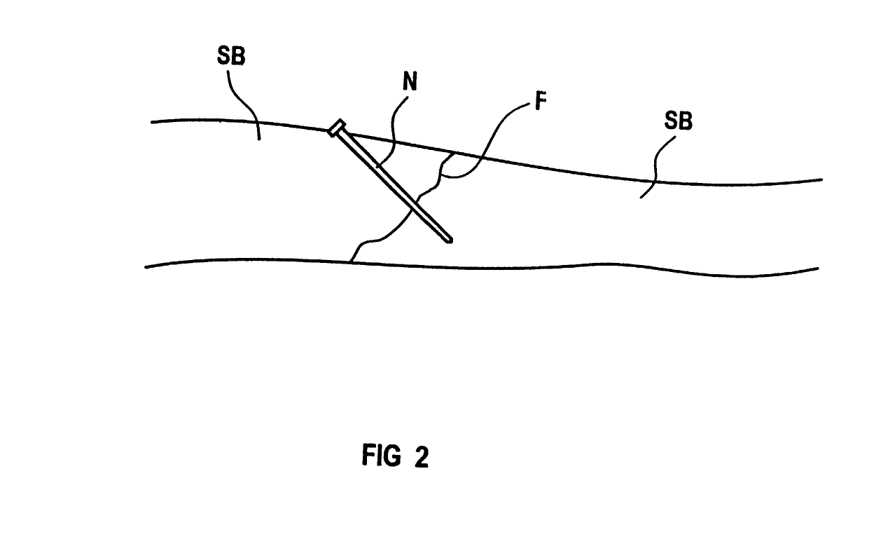

In the exemplary embodiment, the patient P has a fracture F at the left shin bone SB that is shown excerpted and schematically in FIG. 2 and that is to be fixed with a nail N. In order to plan the position of the nail N, an operator (not shown) uses the C-arm X-ray apparatus 1 to generate a volume dataset of the region of interest of the left shin bone SB. Using known methods, for example the MPR (multi-planar reformation) method, the computer 11 produces 2D images of the left shin bone SB in a first operating mode and 3D images of the left shin bone SB in a second operating mode, these being displayable on a monitor 20 that is connected to the computer 11 with an electrical line 21.

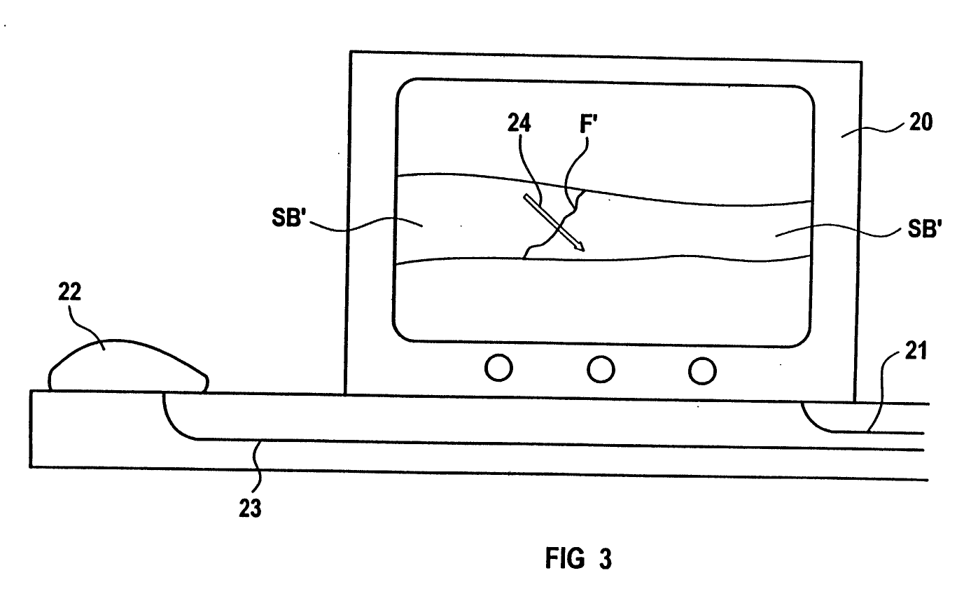

As an example, FIG. 3 shows an image SB′ of the broken left shin bone SB displayed on the monitor 20. In the exemplary embodiment, the image SB′ is a 2D image, and the picture of the fracture F of the left shin bone SB is referenced F′ in FIG. 3.

In the exemplary embodiment with a computer mouse 22 that is connected to the computer 11 with an electrical line, an arrow-shaped marking 24 that corresponds to the future position and alignment of the nail N is set in the image SB′.

After setting the marking 24, the computer 11 assigns a position in the patient to the marking 24 based on the volume dataset, the nail N to be arranged at the shin bone SB of the patient P at said position. Subsequently, the computer 11 adjusts the C-arm 8 with the distance sensors 14 and 15 and the electrical drives 12 and 13 so that a laser beam 26 proceeding from a laser light sighting device 25 arranged at the C-arm 8 characterizes a location 27 at the patient P at which the nail N for fixing the shin bone SB can be introduced so that the nail N assumes the position that corresponds to the marking 24 set in the image SB′. The laser beam also indicates the angle at which the nail N should be introduced.

Instead of the computer mouse 22 shown in FIGS. 1 and 3, other means can be employed for setting a marking in the image. In particular, a track ball or a light pen are also useable.

A viewing device other than the monitor 20 shown in FIGS. 1 and 3 can also be employed. A touch screen can be used which unites the functionality of a viewing device and the functionality of setting a marking in the image.

The image SB′ can also be a 3D image. Markings other than the marking 24 shown in FIG. 3 are also conceivable.

Other means for fixing the bone are also possible, for instance screws or Kirschner wires.

The X-ray device need not necessarily be a C-arm. Other X-ray devices are also possible, as are other means for generating a volume data set that do not make use of an X-ray system. Other means for generating a volume dataset can, for example, be based on ultrasound.

The arrangement for characterizing a location at the examination subject need not necessarily be a laser light sighting device 25. Other optical sighting devices can be employed.

The inventive method or the inventive apparatus also can be utilized for treating other bones or body parts or can be employed for treating animals.

The inventive method or the inventive apparatus also can be utilized for non-medical purposes.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for characterizing a location at a subject, comprising the steps of:
(a) generating a volume dataset of a subject;
(b) generating an image from said volume dataset;
(c) marking a location in said image with a mark; and
(d) based on the mark in said image, adjusting a location characterizing unit relative to said subject so that said location characterizing unit characterizes a location in said subject substantially corresponding to the location in the image identified by said mark.

2. A method as claimed in claim 1 wherein step (a) comprises generating said volume dataset with an X-ray system.

3. A method as claimed in claim 2 comprising moving said X-ray system with at least one drive to generate said volume dataset.

4. A method as claimed in claim 3 comprising moving said X-ray system with at least one electric motor, as said drive, to generate said volume dataset.

5. A method as claimed in claim 3 comprising automatically moving said X-ray system with at least one drive to generate said volume dataset.

6. A method as claimed in claim 1 wherein step (b) comprises generating said image from said volume dataset using a computer, and wherein step (a) comprises generating said volume dataset using said computer.

7. A method as claimed in claim 1 wherein step (b) comprises selecting said image that is generated from the group consisting of two-dimensional images three-dimensional images.

8. A method as claimed in claim 1 comprising the additional step of displaying said image on a viewing device.

9. A method as claimed in claim 1 wherein step (c) comprising marking said location in said image with a marking device selected from the group consisting of a computer mouse, a track ball, a joystick, a light pen, and a touch screen.

10. A method as claimed in claim 1 wherein step (d) comprises adjusting said location characterizing unit with a drive.

11. A method as claimed in claim 10 comprising adjusting said location characterizing unit with an electric motor, as said drive.

12. A method as claimed in claim 10 comprising using said drive, automatically aligning the location characterized by said location characterizing unit with said mark.

13. A method as claimed in claim 1 wherein step (d) comprises characterizing said location at said subject with an optical sighting device, as said location characterizing unit.

14. A method as claimed in claim 13 comprising emitting an optical beam from said optical sighting device to characterize said location at said subject.

15. A method as claimed in claim 14 comprising emitting a laser beam from said optical sighting device to characterize said location.

16. A method as claimed in claim 1 wherein step (a) comprises generating said volume dataset with a C-arm X-ray imaging system.

17. A method as claimed in claim 16 comprising moving said C-arm X-ray imaging system with respect to at least one of an angulation axis and an orbital axis to generate said volume dataset.

18. A method as claimed in claim 16 comprising mounting said location characterizing unit at said C-arm X-ray imaging system.

19. A method as claimed in claim 18 wherein step (d) comprises moving said C-arm X-ray imaging system, with said location characterizing unit mounted thereon, to adjust said location characterizing unit.

20. An apparatus allowing a location at an subject to be characterized, comprising:
an arrangement for generating a volume dataset of a subject;
an arrangement for generating an image from said volume dataset;
a marking arrangement for setting a mark in said image which identifies a location in said subject represented in said image; and
a location characterizing unit which interacts with said marking arrangement to characterize a location at said subject substantially corresponding to the location represented in said image identified by said mark.

21. An apparatus as claimed in claim 20 wherein said arrangement for generating a volume dataset is an X-ray system.

22. An apparatus as claimed in claim 20 wherein said arrangement for generating a volume dataset includes data-generating components, and at least one drive for moving said data-generating components.

23. An apparatus as claimed in claim 22 wherein said drive is an electric motor.

24. An apparatus as claimed in claim 22 wherein said data-generating components are automatically moved by said drive.

25. An apparatus as claimed in claim 20 wherein said arrangement for generating an image from the volume dataset is a computer, and wherein said arrangement for generating a volume dataset also comprises said computer.

26. An apparatus as claimed in claim 20 wherein said arrangement for generating an image from said volume dataset generates said image from the group consisting of two-dimensional images and three-dimensional images.

27. An apparatus as claimed in claim 20 wherein said arrangement for generating an image from the volume dataset includes a viewing device on which said image is displayed.

28. An apparatus as claimed in claim 20 wherein said marking arrangement comprises a marking device selected from the group consisting of a computer mouse, a track ball, a joystick, a light pen, and a touch screen

29. An apparatus as claimed in claim 20 comprising a drive connected to said location characterizing unit for moving said location characterizing unit.

30. An apparatus as claimed in claim 29 wherein said drive is an electric motor.

31. An apparatus as claimed in claim 29 wherein said drive automatically aligns said location characterizing unit to characterize said location substantially corresponding to the location marked in the image.

32. An apparatus as claimed in claim 20 wherein said arrangement for characterizing a location is an optical sighting device.

33. An apparatus as claimed in claim 32 wherein said optical sighting device emits an optical beam to characterize said location at said subject.

34. An apparatus as claimed in claim 33 wherein said optical sighting device is a lower sighting device which emits a laser beam.

35. An apparatus as claimed in claim 20 wherein said arrangement for generating a volume dataset comprises data-generating components mounted on a C-arm.

36. An apparatus as claimed in claim 35 wherein said C-arm is movable relative to at least one of an angulation axis and an orbital axis to generate said volume dataset.

37. An apparatus as claimed in claim 35 wherein said location characterizing unit is mounted at said C-arm.

38. An apparatus as claimed in claim 37 wherein said C-arm is automatically moved, together with said location characterizing unit mounted thereon, to adjust said location characterizing unit.
